(19) United States
(12) Patent Application Publication
(10) Pub. No.: US 2005/0209681 A1
(43) Pub. Date: Sep. 22, 2005

(54) Stent for endoluminal delivery of active principles or agents

(76) Inventors: Maria Curcio, Saluggia (Vercelli) (IT); Enrico Pasquino, Marentino (Torino) (IT); Giovanni Rolando, Chivasso (Torino) (IT); Andrea Grignani, Chieri (Torino) (IT)

Correspondence Address: POPOVICH, WILES & O'CONNELL, PA;650 THIRD AVENUE SOUTH, SUITE 600, MINNEAPOLIS, MN, 55402, US

(21) Appl. No.: 11/051,645

(22) Filed: Feb. 4, 2005

(30) Foreign Application Priority Data
Feb. 5, 2004 (IT) TO2004A000056

(51) Int. Cl. A61F 2/06

(52) U.S. Cl. 623/1.15

(57) ABSTRACT

The present invention provides a stent for implantation at a site within a human or animal body comprising: an expandable body having an inner surface and an outer surface; and treatment agents applied to the outer surface of the expandable body, the treatment agents comprising a combination of Paclitaxel and FK506 or their derivatives or analogues.

### FIELD OF THE INVENTION

The present invention relates to endoluminal delivery of active principles or agents and, in particular, to the delivery of such active principles or agents on a stent.

### BACKGROUND OF THE INVENTION

The term “stent” is intended to include expandable endoprostheses that can be implanted in a lumen of the human or animal body, such as, for example, a blood vessel, to re-establish and/or maintain patency thereof. Stents are usually configured as devices comprising a tubular body which operate so as to maintain a segment of a blood vessel or of another anatomical lumen open.

Stents have become widely used over the last few years for the treatment of stenosis of an arteriosclerotic nature in blood vessels, such as the coronary arteries. Stents are also used in other vessels including, for example, in the dilation of the carotid or peripheral arteries.

The scientific and technical literature, including the patent literature, regarding stents is extremely extensive. For example, the following documents relate to stents: EP-A-0 806 190, EP-A-0 850 604, EP-A-0 875 215, EP-A-0 895 759, EP-A-0 895 760, EP-A-1 080 738, EP-A-1 088 528, EP-A-1 103 234, EP-A-1 174 098, EP-A-1 212 986, EP-A-1 277 449, EP-A-1 310 242, and EP-A-1 449 546.

The activities of research, development and industrial production of stents were directed, in the early years, principally to the geometrical structure and to the corresponding techniques of fabrication (winding of a wire, cutting starting from a microtube, use of superelastic materials, etc.). The research activity regarding stents then gradually extended to particularities of stent fabrication, and in particular to the possibility of applying on the stent or, in some way, associating to the stent, substances having the nature of drugs, and hence able to perform a specific activity on the site of implantation of the stent. See, for example: EP-A-0 850 604, EP-A-1 080 738, and EP-A-1 103 234.

EP 0 850 604 A2 describes the possibility of providing the stent with channels or grooves comprising, for example, cavities that may receive one or more drugs useful in the prevention or in the treatment of restenosis, and/or other substances appropriate for a correct use of the stent (adhesion, modalities of release of the active principle, kinetics, etc.). The surface grooves are characterized, by the shape of their boundary, by the surface of the cavity, and by their profile in depth. For example, the cavities can be cavities with circular or ovoid or elongated openings. Alternatively, they can assume the form of an appropriate alternation of cavities with openings of different types according to the requirements of drug release. The depth profile can be U-shaped or V-shaped, or shaped like a vessel, with or without a surface part completely dedicated to receiving the substances of interest referred to above. The surface part can assume the appearance of a sort of continuous layer on just the outer surface of the stent. See, for example: WO-A-98/23228, EP-A-0 950 386, and EP-A-1 277 449.

The stent described in EP 1 277 449 A1 envisages that in the elements of the reticular structure of the stent there will be provided cavities that can function as true reservoirs for receiving agents for the treatment of the site of implantation of the stent. Where present, the cavities bestow upon the respective element a hollowed profile, the cavities occupying a substantial portion thereof. The geometry of the cavities is chosen in such a way as to leave substantially unimpaired the characteristics of resistance to bending of the respective element. The cavities provided in this stent also enable the quantity of agent associated to the stent to be sufficient even when release over a prolonged period of time is desired and even though the surfaces of the stent, and, in particular, the internal surface, are subjected to an action of flushing by the blood flow. Furthermore, the geometry of this stent and its cavities enables the active or activatable agent to be made available and released prevalently, if not exclusively, on the outer surface of the stent and not on the internal surface thereof. This is significant particularly in the case where the agent applied on the stent has to perform a restenosis-antagonist function. In that situation the corresponding mechanism of action, which aims at acting on the outer surface of the stent facing the wall of the treated vessel, could in fact have adverse effects if it were applied on the internal surface of the stent. For example, it could hinder the phenomena of formation of neointima on the internal surface of the stent, which is considered beneficial in the post-implantation phase.

The configuration of the stent described above makes it possible to have available stents capable of being configured as true vectors of active or activatable agents, possibly different from one another, made available in sufficient quantities to achieve a beneficial effect that is also prolonged in time. Additionally, the agents may even be different from one another, located selectively in different positions along the development of the stent. This enables the dosages to be selectively varied in a localized way, for example, achieving differentiated dosages in the various regions of the stent.

The stent and drug delivery mechanisms described in the patent documents cited previously respond primarily to requirements linked to the mechanism of release of the active agent including: (i) the quantity of agent that may be released, (ii) the position in which the agent (or the various agents) arranged on the stent are released, and, albeit in to a lesser extent, (iii) the kinetics of release of the active agent.

The present invention includes the identification of pharmacologically active compounds, or, again more preferably, of associations of pharmacologically active compounds to be delivered via a stent and that are to perform an effective restenosis-antagonist function.

It is known that, in a relatively large proportion of patients to which a stent has been applied, a new stenosis develops. It has been discovered that this so-called restenosis is generated by a new formation of the vascular architecture of the layers of tissue. In particular, the introduction of a stent in the stenotic site can result in damage to the tissues of the blood vessel (generally referred to as “mechanically mediated vascular injury”) with consequent inflammatory reactions, hyperproliferation, and migration of smooth muscle cells (SMCs) into the damaged stenotic site. In the model of animal restenosis and also in human tissue it has been found that the hyperproliferation of SMCs is accompanied by infiltration of the tissue around the reinforcements of the stent by macrophages and T-cells, as is for example described in Grewe et al., J. Am. Coll. Cardiol. 35 (2000), 157-63.

Research activity has concentrated on the identification of pharmacologically active substances, in particular with the aim of providing a stent with a capacity for release in situ of one or more of these substances, exploiting possible synergistic or diversified reactions. Specific attention has been paid to the role played by certain drugs in regard to inflammatory and/or hyperproliferative reactions. In addition to pharmacologically active molecules, the stent can carry substances with a function of adjuvant of the pharmacologically active substances, such as polymers or excipients of various nature. The function of the latter may be stabilization of the active principles, or may be aimed at regulating the kinetics of release (deceleration or acceleration of release). The polymers/excipients can be mixed with the drug or drugs or can be in separate layers with respect to the pharmacologically active substances, for example, forming in the context of a cavity a sort of operculum of bio-erodible polymer, i.e., creating a stratified structure, with successive layers of drug and polymer. These activities are documented extensively in the scientific and patent literature which includes, in addition to some of the documents cited previously, the following: EP-A-0 551 182, EP-A-0 747 069, EP-A-0 950 386, EP-A-0 970 711, EP-A-1 254 673, EP-A-1 254 674, WO-A-01/87368, WO-A-02/26280, WO-A-02/26281, WO-A-02/47739, WO-A-02/056790, WO-A-02/065947, WO-A-01/87372, WO-A-01/87375, WO-A-95/03036, as well as by the literature cited in these documents.

As regards the choice of the drug with restenosis-antagonist function, the drugs proposed for that purpose are extremely numerous. Even more numerous, even though not fully described,, are the hypothetical combinations of two or more drugs belonging to the same class or to different classes. The drugs proposed comprise, for example: anti-inflammatory drugs of the corticosteroid type (Cortisol, Betamethasone, Fluocinolone, Cortisone, Dexamethasone, Fluocinonide, Corticosterol, Flunisolide, Fluoromethalone, Tetrahydrocortisol, Aldlomethasone, Flurandrenolide, Prednisone, Amcinonide, Alcinonide, Prednisolone, Clobetasol, Medrisone, Methylprednisolone, Clocortolone, Momethasone, Fluodrocortisone, Desonide, Rofleponide, Triamcinolone, Desoxymethasone, Paramethasone, Diflorasone); anti-inflammatory agents of the non-steroidal type (Acetylsalicylic acid, Diflunisal, Salsalate, Phenylbutazone, Oxyphenbutazone, Apazone, Indomethacin, Sulindac, Mefenamic acid and fenamates, Tolmetine, Ibuprofen, Naproxen, Fenoprofen, Ketoprofen, Flurbiprofen, Piroxicam and derivatives, Diclofenac and derivatives, Etodolac); and anti-neoplastic, anti-proliferative, and/or immunosuppressive agents (Cyclophosphamide, Melphalan, Chlorambucil, Ethyleneimine and Methylmelamine, Alkyl sulphonates, Nitrosoureas, Triazines, Metotrexate, Fluorouracil, Mercaptopurine, Thioguadinine, Vinblastine, Vincristine, Etoposide, Actinomycin D, Doxorubicin, Cisplatin, Mitoxantrone and derivatives, Hydroxyurea and derivatives, Procarbatine and derivatives, Mitotanes, Aminoglutetimide, Docetaxel, Paclitaxel and analogues, 7-hexanoyl-taxol, Epothilones, Batimastat and analogues, Rapamycin and analogues, FK506 (tacrolimus), Cyclosporine).

Some of the compounds cited above have been described in constituted patent publications including: WO-A-02/065947 and EP-A-1 254 674 regarding the use of FK506 (an immunosuppressive drug) and WO-A-01/87372, WO-A-01/87375, and WO-A-95/03036 regarding the use of Paclitaxel (anti-proliferative agent).

WO-A-02/065947 describes a stent which is loaded with FK506 possibly in combination with other active substances, where the list of the possible additional drugs comprises approximately sixty compounds. The modalities of application of the drug to the stent envisage that the stent will be brought into contact with a solution of FK506 in aqueous or organic solvent (typically in alcohol) for example, by means of dripping, spraying, or immersion, preferentially under a vacuum. The stent is then dried, preferably up to total removal of the solvent, the operation being repeated from 1 to 5 times. Subsequently, the stent is possibly washed with water or isotonic saline solution and then dried again. It has, however, been noted that the technical solution described WO-A-02/065947 may not be effective in the treatment of restenosis. It may be hypothesized that this derives from the fact that the amount of drug, and in particular of FK506, loaded on the stent following the method described herein, is not pharmacologically active in regard to the cell processes that underlie restenosis, presumably because it is smaller than the threshold value of therapeutic effectiveness.

This result is corroborated also by the scientific literature that has experimented with FK506 in cultures of SMCs in view of inhibition of the proliferation of smooth muscle cells (Mohacsi et al., J. Heart Lung Transplant. 16 (1997) 484-492; Marx et al., Circulation Res., 76 (1995) 412-417), and of their migration (Poon et al., J. Clin. Invest. 98 (1996) 2777-2283). In general, FK506 has been deemed unsuitable for the prevention of restenosis on account of its low power, as witnessed by Mohacsi et al., J. Heart Lung Transplant. 16 (1997) 484-492; Poon et al., J. Clin. Invest. 98 (1996) 2277-2283; Marx et al., Circulation Res., 76 (1995) 412-417; Dell, Curr. Med. Chem. 5 (1998) 179-94. Ranamycin (another immunosuppressive drug) has proven, instead, active in the inhibition of the proliferation of SMC cultures.

EP 1 254 674 A1 identifies quantities by weight of FK506 usable on a stent as restenosis-antagonist agent. This description is, however, ambiguous in so far as the quantities of drug indicated to be used are alternatively expressed in milligrams, micrograms, and picograms, always in relation to a stent of 16 mm in length. Such a description does not therefore indicate a therapeutically effective concentration of FK506 as a restenosis-antagonist agent, nor does it enable such a concentration to be deduced.

WO-A-01 87372, WO-A-01 87375, and WO-A-95 03036 describe the use of the anti-proliferative drug identified as Paclitaxel or Taxol, a substance with cytostatic, anti-proliferative, and/or anti-angiogenic activity, applied directly or indirectly on a stent, once again for the purpose of acting as restenosis-antagonist.

In regards to the use of the drug, possibly in combination with another pharmacologically active compound, such as for example Rapamycin, there are numerous scientific articles including: Herdeg et al., Semin. Intervent. Cardiol. 3 (1998) 197-199; Hunter et al., Adv. Drug Delivery Rev. 26 (1997) 199-207; Burke et al., J Cardiovasc. Pharmacol., 33 (1999) 829-835; Gallo et al., Circulation 99 (1999) 2164-2170, according to which the results of the application of the association of Paclitaxel in combination with Rapamycin in laboratory animals is not effective in the treatment of restenosis and even harmful in so far as it would seem to lead to inhibition of the formation of neointima. It has moreover been observed that after six months from insertion in pigs of stents coated with Paclitaxel, there was found a disappearance of the effect (Heldman, International Local Drug Delivery Meeting and Cardiovascular Course on Radiation, Geneva, Jan. 25-27, 2001).

Rapamycin is believed by many to be the drug with the best potentiality of application for an almost complete elimination of restenosis, as supported by the first clinical findings (Sousa et al., Circulation 103 (2001) 192-195). On the other hand, the use of the Rapamycin, according to some scientists, would seem to give rise to a decelerated healing of the vascular wall injured by balloon angioplasty and insertion of the stent. It is thus very important to achieve a balance between treatment of the arterial vascular wall after angioplasty and insertion of the stent, on the one hand, and the formation of neointima on the internal wall of the stent facing the blood flow, on the other.

### SUMMARY OF THE INVENTION

One of the objects of the present invention is to make available implants with properties favorable for the treatment of restenosis and capable of reducing undesirable effects to the minimum. According to the present invention, this object is achieved according to a stent having the characteristics referred to specifically in the ensuing claims.

The solution described herein is based upon the observation of the fact that the target of a truly effective restenosis-antagonist action can be achieved via identification of a combination of drugs and more preferably through identification of the correct ratios (concentrations) between the two drugs of the combination in order to reduce to the minimum their secondary effects and increase as much as possible their pharmacological activity.

Furthermore, the present applicants have verified that the modalities of loading of the association of drugs on the stent are important. It is, in fact, preferable that the drugs should be free, i.e., that they should be applied without prior dissolution or suspension as commonly envisaged in the known art, where techniques of dripping, immersion, or spraying, on the stent, of a solution containing the active principle dissolved in a solvent are adopted.

In a preferred embodiment, the present invention provides a stent to which there are associated Paclitaxel and FK506 or their derivatives or analogues in combination, appropriately loaded within the cavities present on the outer surface of the stent, and optionally also on the entire outer surface of the stent, in a Paclitaxel:FK506 weight ratio with respect to the total quantity of said agents loaded on the stent comprised in the range between 1:72 and 1:0.2.

### BRIEF DESCRIPTION OF THE DRAWINGS

The invention will now be now described in a detailed way, purely by way of non-limiting example, with reference to the annexed drawings, in which:

FIG. 1 is a schematic illustration of the cross section of a stent usable in the context of the present invention;

FIGS. 2 to 7 are schematic illustrations of different embodiments of the stent according to the invention; and

FIG. 8 illustrates the embodiment of an operation of loading of a stent using a device for dispensing a paste.

### DESCRIPTION OF THE PREFERRED EMBODIMENTS

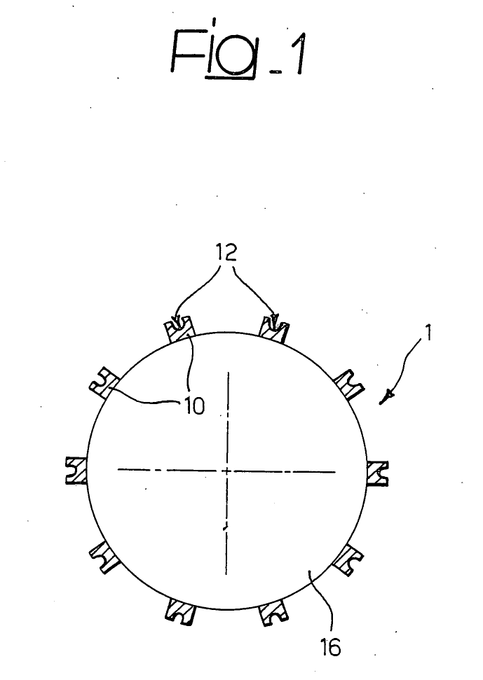

The present description is provided, purely by way of non-limiting example, with reference to a stent 1 as shown in FIG. 1, and substantially corresponding to the stent described in U.S. Pat. No. 6,325,821 B1, the contents of which are hereby incorporated by reference herein. Such a stent is constituted by a tubular body made of metallic material, that may be dilated starting from a radially contracted condition into a radially expanded condition. The body of the stent comprises a plurality of structural elements or “struts” 10, which define an openwork structure as a whole of reticular nature. In particular, in the stent described in U.S. Pat. No. 6,325,821 B1, the structure in question is described as comprising a plurality of annular segments arranged in succession along the longitudinal axis of the stent. The segments in question have a serpentine pattern with the bend parts arranged in opposite sequence, connected together by connecting elements, commonly referred to as “links”. The serpentine sequences of the successive segments are usually in phase opposition, i.e., with a concavity of each serpentine opposite to a concavity of an adjacent segment. The links have a substantially V-shaped pattern with a profile characterized by an alternation of concave portions and convex portions. The aforesaid links connect the various annular segments of the stent at the “0” points of the serpentine paths of the segments. It should be noted, however, that the present invention is equally applicable to stents having differing geometries.

In FIG. 1 the stent 1 is represented in cross section, so that there appears only the circular trace of cross section defined by a certain number of struts 10 traversed by the plane of cross section. The stent in question is provided on its outer surface with grooving formed by a pattern with cavities 12 of the type described in EP 0 850 604 or in EP 1 277 449. The cavities 12 can receive within them respective amounts of a substance constituted by Paclitaxel and FK506, according to the modalities described hereafter.

In a particularly advantageous way, the stent in question may be coated on its outer surface by a layer of biocompatible carbon material deposited thereon, by applying the technique described, for example, in U.S. Pat. Nos. 5,084,151; 5,133,845; 5,370,684; 5,387,247; and 5,423,886. The presence of said coating of biocompatible carbon material proves advantageous for the purposes of use of the stent, in particular as regards the minimization of the effects mediated by implantation of the stent.

In a preferred embodiment, the method for carrying out the loading/application of drug or active principles or agents on a stent according to the present invention starts from powders or pastes obtained from the powders of the two drugs Paclitaxel and FK506 possibly mixed together, where the two drugs have a Paclitaxel:FK506 weight ratio, with respect to the total quantity of drugs loaded on a stent, comprised in the range from 1:72 to 1:0.2, preferably from 1:18 to 1:0.2, more preferably from 1:8 to 1:0.5, still more preferably from 1:2 to 1:0.7, and to a more preferred extent from 1:3 to 1:1.

With reference to a stent for coronary angioplasty having a length of approximately 15 mm and a diameter in the expanded condition of approximately 3.0 mm to 3.5 mm, the present applicants have reason to believe that a total quantity of drugs, Paclitaxel and FK506, loaded/applied on the stent, without considering the possible presence of additives and/or excipients, comprised in the range between 50 and 1000 micrograms may be therapeutically effective. Preferably, the total quantity of Paclitaxel and FK506 loaded/applied on the stent is in the range between 70 and 700 micrograms, more preferably the quantity may be comprised between 170 and 500 micrograms, and still more preferably between 200 and 400 micrograms. It is evident that when reference is made to loading/application on the stent of these drugs, this may mean both the loading within the cavities alone present on the outer surface of the stent and a generalized loading on the entire outer surface of the stent, including loading also within the cavities.

The present applicants, once again on the basis of evaluations relating to a stent for coronary angioplasty having a length of approximately 15 mm and a diameter in the expanded condition of approximately 3.0 mm to 3.5 mm, have reason to believe that the minimum therapeutically effective quantity of Paclitaxel, when this drug is in combination with FK506, is greater than 25 micrograms and more preferably greater than 80 micrograms.

Without limitation to any theory in this regard, it is believed that the therapeutic effectiveness of the one active agent is supported rather than integrated by the presence of the other active agent comprised in the combination, even though the other active agent is present in a minimum amount and possibly less than the threshold value of therapeutic effectiveness reported in the literature. In other words, the two agents, in combination, have a synergistic effect. It may be hypothesized, in fact, that in these conditions the combined therapeutic effect of the two drugs is exploited directly on the stenotic site, i.e., the direct application of the two drugs on the stenotic blood vessel, in which implantation of the stent has been performed to re-establish and/or maintain its patency, will determine a tissue concentration of the two drugs sufficient to achieve therapeutic effectiveness.

In a way similar to what has been described above in relation to Paclitaxel, the present applicants have reason to believe that the minimum amount of FK506 therapeutically effective in the stenotic site, when said drug is applied in combination with Paclitaxel, is greater than 60 micrograms and more preferably greater than 135 micrograms.

It should be recalled that FK506 is the macrolide antibiotic FK506 (Tacrolimus, [3S-[3R*[E(1S*,3S*,4S*)],4S*, 5R*,-8S*,9E,12R*,14R*,15S*,16R*,18S*,19S*, 26aR*]]-5,6,8,11,12,13,14,15,16,17,18,19,24,25,25,26a-hexadecahydro-5,19-dihydroxy-3-[2-(4-hydroxy-3-methoxycyclohexyl)-1-methylethenyl]-14,16-dimethoxy-4,10,12,18-tetramethyl-8-(2-propenyl)-15,19-epoxy-3H-pyrido[2,1-c][1,4]oxa-aza-cyclotricosine-1,7,20,21(4H,23H)-tetrone; Merck index No. 9000). It is an active substance which was originally developed for the medicine of transplants and the immunosuppressive action of which is considered to extend also to the restenosis-antagonist mechanism.

Paclitaxel belongs to the family of the diterpenoids and has the scientific name [2aR-[2aα,4β,4aβ,6β,9α-(αR*,βS*),11α,12α,12aα,12bα]]-β-(benzoylamino)-α-hydroxy-benzene propanoic acid 6,12-bis(acetyloxy)-12-(benzoyloxy)-2a,3,4,4a,5,6,9,10,12,12a,12b-dodecahydro-4,11-dihydroxy-4a,8,13,13-tetramethyl-5-oxo-7,11-methano-1H-cyclodeca-[3,4]benz[1,2-b]oxet-9-yl ester, Merck index No. 7052. Paclitaxel is extracted from the bark of Taxus brevifolia and is produced by Taxomyces Andreanae, an endophytic fungus of Taxus brevifolia (Stierle et al., Science 260, 214-216, 1993). Paclitaxel is distributed commercially under the trade name TAXOL® by Bristol Myers Squibb. Paclitaxel exerts an anti-proliferative and anti-angiogenic effect by acting on the intracellular microtubules.

Without limitation to any specific theory in this regard, the present applicants have reason to believe that the use of the association of the two drugs or of their derivatives or analogues, above all in the particular conditions of application, i.e., of concentration of the two drugs with respect to one other, will lead to Paclitaxel being able to act as an inhibitor of proliferation of SMCs, altering the dynamic balance between microtubules and α- and β-tubulin, favoring the formation of abnormally stable microtubules. Paclitaxel in this way interferes with the capacity of the cell to maintain its shape and directionality of movement, to transmit intracellular signals and to carry out intracellular transport. FK506 is believed to be able to explicate its immunosuppressive action by contrasting the presence of macrophages and T-cells in the implantation site, and it is also believed that it may be able to act as an anti-inflammatory agent, mitigating the inflammatory action induced by the procedure of angioplasty and insertion of the stent, enabling the normal phenomenon of healing/cicatrization of the wall of the vessel.

It has moreover been verified that the application in the form of powders or of pastes formed from said powder manages to reconcile the need to provide a selective application (an important factor both for the purposes that it is intended to pursue and for the specific cost, which is usually rather high, of the substances applied), with the need to ensure firm retention of the substances applied on the stent. The specific details of the application of said powders or pastes are described in U.S. patent application Publication No. US 2004/0167612 A1, the contents of which are hereby incorporated by reference herein.

It will be appreciated that the term “paste” is intended herein to indicate any plastic mass having a degree of viscosity such as to cause said mass to conserve substantially its shape if exposed only to the force of gravity. The above is in evident contrast with a liquid or cream, which does not present said characteristic of conservation of shape.

The solution described herein envisages that there may be added to the two drugs, either mixed therewith or in separate layers, excipients and/or polymers having the purpose of agents for filling the cavities 12 set on the outer surface of the stent or of agents having the capacity of controlling (i.e., accelerating or decelerating) release of the drugs.

An embodiment of the solution described herein is illustrated in FIG. 2, which shows just one strut provided with a cavity 12 that is to be filled with a mixture of the two drugs 5, where the mixture 5 is constituted by Paclitaxel and FK506 in a weight ratio with respect to the total amount of drugs loaded on the stent, without considering the presence of possible excipients and/or additives, comprised between 1:72 and 1:0.2, advantageously between 1:18 and 1:0.2, more preferably between 1:8 and 1:0.5, still more preferably between 1:2 and 1:0.7, and to an even more preferred extent between 1:3 and 1:1. This embodiment exploits the effect linked to the simultaneous release of the two drugs. The mixture, as already described previously, can contain possible excipients.

The process of loading of the mixture can be selective in the sense that it concerns only those areas of the stent that effectively carry the drugs, as well as the possible additives (excipients). Of course, this result can be achieved also by way of a generalized loading, initially involving also areas of the stent that are then to be cleaned from the excess of the substances loaded.

Once the chosen substances have been deposited on the stent (before or after cleaning, according to the operating choices or the techniques adopted), the next step is fixation (stablization) of the drugs, as well as of the additives. The purpose of this is to ensure that the substances may be effectively carried/delivered by the stent onto an implantation site and not be dispersed. elsewhere, in particular during advance towards the implantation site or even before the stent is inserted into the body in which it is to be implanted.

At least in principle, the operations of stablization can be conducted in a selective way only on the parts where the presence of the substances is required or else in a generalized way on the entire stent, at least as regards the outer surface thereof. Specifically, in the case where the process starts from powders, the main methods of loading that can be adopted are: (1) recourse to a non-selective corona effect (electrostatic effect), i.e., coating all the surface with powder and then cleaning the areas that require cleaning; (2) recourse to a selective electrostatic process (such as a photocopier or laser printer), for example through an intermediate roller that collects the powder only in areas corresponding to the cavities where it deposits the powder subsequently; if the stents are ones having the entire outer surface porous, in general selectivity is not necessary, and no specific operation of cleaning may be required; (3) rolling on a bed or mat of powder, with subsequent cleaning; and (4) rolling on a bed or mat of powder with a mask of a semigraphic type to load just the cavities; cleaning is not required.

In the case where the process starts from a paste, the main methods of loading that can be adopted are: (1) rolling on a bed or mat of paste, with subsequent cleaning; (2) rolling on a bed or mat of paste with a protective mask (serigraphy); cleaning is not required; and (3) application with a dispensing nozzle, typically driven by a fine-positioning numeric-control machine.

It will be appreciated that whatever the modalities of loading adopted, for pastes or for powders, it is then preferable to proceed to an operation of stablization. The term “stablization” means to bestow upon the contents of the cavities a mechanical stability and a degree of adhesion to the cavities themselves adapted to the mechanical stresses undergone by the stent during transportation, stockage, procedures of insertion into the body, and expansion.

To achieve the desired effect of stablization it is possible to resort to techniques such as: (1) exposure to temperature or thermal cycles; (2) immersion in solvent for controlled lengths of time; (3) exposure to solvent sprays; (4) exposure to solvent vapors; (5) selective treatment with laser (exposure of the cavities to a laser beam); (6) selective or integral application of an adhesive protective coating (as an operculum for closing the cavities); and (7) lyophilization.

It will likewise be appreciated that the final result achieved is the placing of the drug or of the mixture on the surface of the stent 1, in a position directly exposed on the outside, even though usually at least slightly retracted inside the cavities 12, without any need to have available coatings/sheaths of some kind on the surface of the stent. The latter thus remains free, with the coating of biocompatible carbon material preferentially provided thereon, with consequent beneficial effects both during implantation of the stent and in the post-implantation phases.

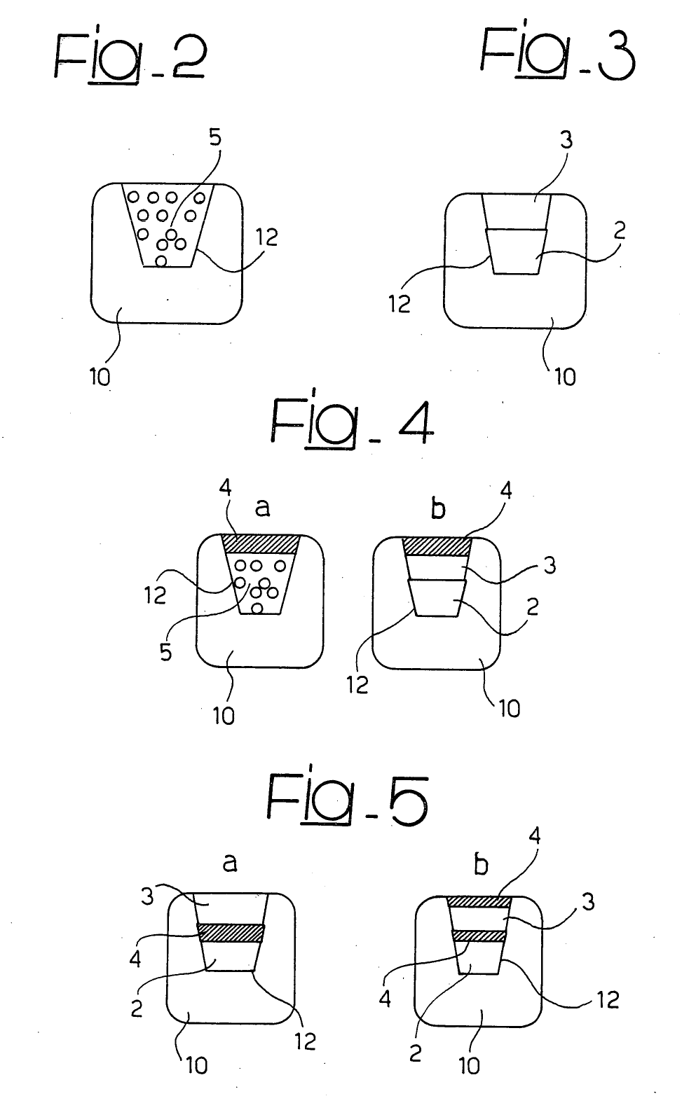

A second example of the solution described herein is shown in FIG. 3. In this embodiment cavity 12 can be filled, according to the modalities described above, in two steps, setting on the bottom of the cavity 12 a first layer 2, constituted by Paclitaxel, and on top a second layer 3, constituted by FK506. Alternatively, the composition of the first layer and the second layer can be reversed so as to have a first layer 2 of FK506 on the bottom of the cavity 12 and on top a second layer 3 of Paclitaxel, or again combining a first layer 2 of a drug (whether it be Paclitaxel or FK506) with a second layer 3 constituted by a mixture of the two drugs or vice versa (layer 2 constituted by a mixture, and layer 3 constituted by a drug), or again both of the layers 2 and 3 constituted by mixtures of the two drugs, but with ratios different from one another, the foregoing once again in due respect of the ratios/quantities by weight between the two drugs described and identified above.

The example illustrated in FIG. 3 exploits the release of the two drugs in different times. In the case where the first layer is constituted by Paclitaxel and the second layer is constituted by FK506, there will initially be an effect (which is likely to be of an anti-inflammatory type) on account of the release of FK506 and only subsequently an effect (which is likely to be of an anti-proliferative type) linked to the release of Paclitaxel. In the case where it was desired to favor initially the anti-proliferative effect and only subsequently an anti-inflammatory effect for promoting final healing of the blood vessel, the first layer 2 could be advantageously constituted by FK506 and the second layer 3 by Paclitaxel, it being evident that it is always the second layer 3 positioned within the cavity 12 to be absorbed first by the walls of the vessel. Even though in this case a mixture of the two drugs is not used, there is even so obtained a combined (therapeutic) effect of the two drugs in the implantation site.

This embodiment involves depositing, in the form of powder or paste, one of the two drugs or a mixture thereof on the bottom of the cavity, followed by a step of stablization of this first layer; only subsequently, is the second drug or mixture of drugs deposited and then stabilized. In the case where stablization envisages the use of a solvent, there could be hypothesized the use of two different solvents for stablization of the two layers for the purpose of preventing undesirable mixing of the two drugs at the interface of contact between the two layers.

In the case where it is necessary, rather than merely appropriate, to provide a non-immediate action of the two drugs after implantation of the stent or else at times regulated with greater precision, controlled release can be obtained via the introduction of layers constituted by polymers having a porous structure, or by bio-erodible polymers, or by excipient substances.

FIG. 4a illustrates a case where the aim is that the layer of mixture 5 set within the cavity should be made available to the organism after a definite period of time t subsequent to the moment of implantation of the stent (understood as time zero). In these cases, the layer of polymer or of excipient substance 4 is applied to be eroded/absorbed by the wall of the vessel in a time t determined by the composition as well as by the thickness of layer 4. Only subsequently will the mixture 5 of two drugs be released. FIG. 4b shows the application of the same principle to a solution in which the two drugs 2 and 3 (whichever the drug constituting each layer) are arranged on top of one another.

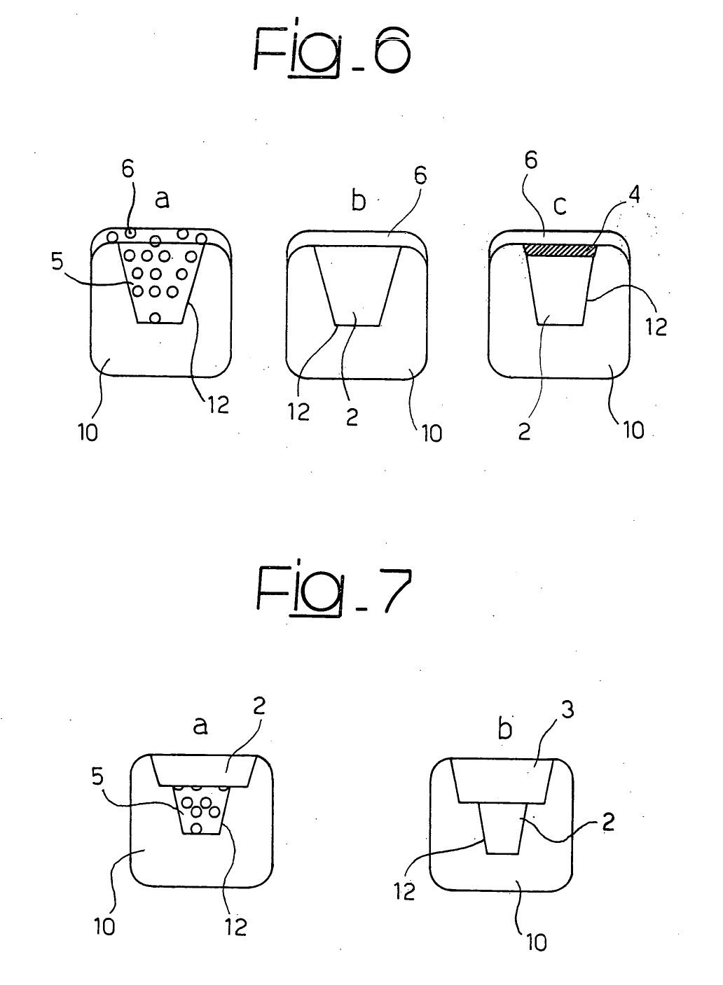

A polymeric layer or a layer of excipient substances 4 can also be used, with the same modalities, for the physical separation of the two layers of drugs 2 and 3 (see FIGS. 5a and 5b) in the case where it is desired to prevent their spontaneous mixing at the interface of contact of the two layers. An example of application of this solution can be represented by the case where the same solvent is used for the stablization of the two drugs deposited within the cavity in two successive steps, where the identity of solvent could favor the destabilization of a surface portion of the layer of a drug already present in the cavity, with subsequent mixing with the powder or paste of the second drug not yet stabilized.

As regards the composition of the bioerodible-polymer-based layer 4, reference can be made to the very extensive literature published on the subject, which is well known to the person skilled in the art. By way of example suitable polymers include: acrylates and methacrylates, silicones, such as for example polydimethylsiloxane, polymethylene malonester, polyethers, polyesters, bio-absorbable polymers, polymers of vinylic monomers, such as for example polyvinyl pyrrolidone and vinyl ether, poly-cis-1,4-butadiene, poly-cis-1,4-isoprene, poly-trans-1,4-isoprene and vulcanized products, polyurethane, polycarbamides, polyamides, polysulphones and biopolymers, such as for example cellulose and its derivatives, proteins, and fibrin bonding agents. Particularly interesting properties are demonstrated by hydrogels, which on account of their high absorption of water have very good haemocompatiblity as the outermost layer (top coat). It is, for example, possible to use hydrogels such as polyacrylamide, polyacrylic acid, polymers with oxygen as heteroatom in the main chain, such as for example polyethylene oxide, polypropylene oxide, and polytetrahydrofuran.

FIGS. 6a, 6b, and 6c show embodiments where the entire outer surface of the stent (and not only within the cavities 12) has applied thereon a homogeneous layer of coating 6 constituted by the mixture of the two drugs or else by just one of the two drugs for the purpose of increasing substantially the therapeutic impact in situ on the biological processes that underlie restenosis.

The top coat 6 of the mixture of the two drugs or of just one of the drugs is performed subsequent to filling of the cavities, once again using a powder or paste of the mixture or of the single drug of interest. The modalities of loading and stablization of the layer of top coat 6 are similar to the ones previously described in relation to the loading of the cavities. In principle, this top coat 6 of the stent could be applied when the stent has already been positioned on the catheter for implantation for the purpose of preventing problems linked to the manipulation of the coated stent during assembly of the stent on the catheter and hence preventing any problem of damage to the coating, with possible loss of a portion thereof. It is evident that the quantity of the two drugs loaded/applied on the stent as illustrated in FIG. 6 always respect the weight ratios rather than the total or individual amounts of the two drugs with respect to one other, as described above.

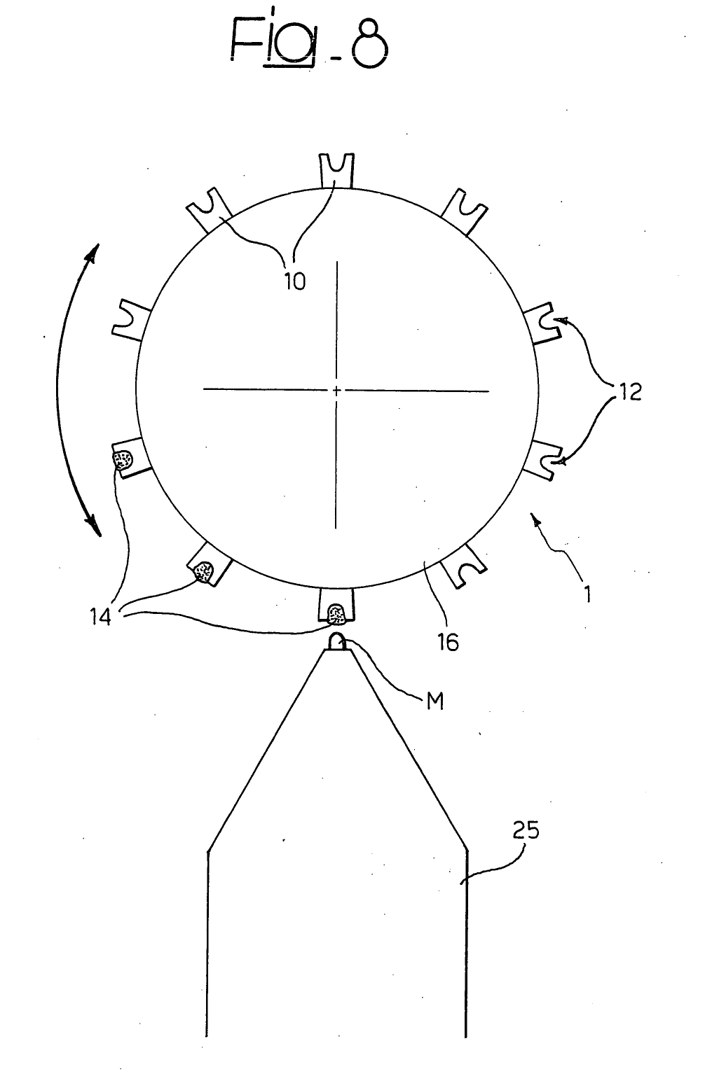

Illustrated in FIG. 7 is an example of an embodiment in which the cavity 12 in the stent 1 has a step-like profile, i.e., a configuration such as to provide further degrees of freedom in the choice of: (i) the quantity of a drug/mixture to be introduced within the cavity; and (ii) the kinetics of release of the drug/mixture, which is obviously controlled by the surface of the opening of the cavity.

FIG. 8 shows a device for applying selectively, and hence only within the cavities 12, respective amounts 14 of material in the form of a paste. The device includes a dispensing nozzle 25, which may be brought into a position facing the cavities 12, bestowing then upon the cavity 12 each time involved and upon the nozzle 25 a relative displacement that leads the nozzle to “scan” the cavity 12, depositing therein the drug/mixture M. This solution may be implemented with high precision by resorting to a numeric-control machine for controlling the relative movement (usually of translation and rotation) of the support 16 that carries the stent 1 and the dispensing nozzle 25.

Without limitation to any specific theory in this connection, the present applicant has reason to believe that the choice of the values of the Paclitaxel:FK506 weight ratio in the ranges from 1:72 to 1:0.2, preferably from 1:18 to 1:0.2, more preferably from 1:8 to 1:0.5, still more preferably from 1:2 to 1:0.7, and to an even more preferred extent from 1:3 to 1:1, is particularly beneficial.

Without prejudice to the principle of the invention, the details of implementation and the embodiments may vary with respect to what is described and illustrated herein, without thereby departing from the scope of the invention, as defined by the annexed claims.

1. A stent for implantation at a site within a human or animal body comprising:
an expandable body having an inner surface and an outer surface; and
treatment agents applied to the outer surface of the expandable body, the treatment agents comprising a combination of Paclitaxel and FK506 or their derivatives or analogues.

2. The stent according to claim 1, wherein the outer surface of the expandable body includes formations for receiving the treatment agents.

3. The stent according to claim 2, wherein the formations comprise cavities having an opening towards the outer surface of the expandable body.

4. The stent according to claim 1, wherein the expandable body is formed by elements defining a reticular structure.

5. The stent according to claim 4, wherein the formations are present on the elements.

6. The stent according to claim 1, wherein the treatment agents are present in a Paclitaxel:FK506 weight ratio with respect to the total quantity of said agents applied on the expandable body comprised in the range from 1:72 to 1:0.2.

7. The stent according to claim 6, wherein the weight ratio is comprised in the range from 1:18 to 1:0.2.

8. The stent according to claim 6, wherein the weight ratio is comprised in the range from 1:8 to 1:0.5.

9. The stent according to claim 6, wherein the weight ratio is comprised in the range from 1:2 to 1:0.7.

10. The stent according to claim 6, wherein the weight ratio is comprised in the range from 1:3 to 1:1.

11. The stent according to claim 1, wherein the treatment agents are present in a total quantity comprised in the range between 50 and 1000 micrograms.

12. The stent according to claim 11, wherein the total quantity is comprised in the range between 70 and 700 micrograms.

13. The stent according to claim 11, wherein the total quantity is comprised in the range between 140 and 500 micrograms.

14. The stent according to claim 11, wherein the total quantity is comprised in the range between 200 and 400 micrograms.

15. The stent according to claim 1, wherein Paclitaxel is present in a quantity by weight of at least 25 micrograms.

16. The stent according to claim 15, wherein Paclitaxel is present in a quantity by weight of at least 80 micrograms.

17. The stent according to claim 1, wherein FK506 is present in a quantity by weight of at least 60 micrograms.

18. The stent according to claim 17, wherein FK506 is present in a quantity by weight of at least 135 micrograms.

19. The stent according to claim 2, wherein the formations for receiving comprise cavities and wherein the cavities contain a homogeneous mixture of the treatment agents.

20. The stent according to claim 2, wherein the treatment agents are arranged in a layered structure in the formations comprising at least two layers.

21. The stent according to claim 20, wherein the layers are homogeneously constituted by one of the treatment agents or by a mixture of the treatment agents.

22. The stent according to claim 20, wherein the layered structure includes an associated layer comprising at least one of a polymeric material and an excipient substance.

23. The stent according to claim 22, wherein the associated layer is situated in an internal position of the layered structure.

24. The stent according to claim 22, wherein the associated layer is situated in a position external to the layered structure.

25. The stent according to claim 1, further comprising a top coat applied to the outer surface comprising one of the treatment agents or a mixture of the treatment agents.

26. The stent according to claim 2, wherein the formations for receiving comprise cavities having a substantially step-like profile.

27. The stent according to claim 1, wherein the treatment agents are applied in the form of powders or pastes obtained from the powders.

28. The stent according to claim 1, further comprising pharmacologically acceptable excipients/additives which are added to the treatment agents.

29. The stent according to claim 1, wherein the treatment agents comprise a combination of Paclitaxel and FK506.

30. The stent according to claim 29, wherein the combination of Paclitaxel and FK506 is present in a total quantity comprised in the range between 50 and 1000 micrograms.

31. The stent according to claim 30, wherein the total quantity is comprised in the range between 70 and 700 micrograms.

32. The stent according to claim 30, wherein the total quantity is comprised in the range between 140 and 500 micrograms.

33. The stent according to claim 30, wherein the total quantity is comprised in the range between 200 and 400 micrograms.

34. The stent according to claim 29, wherein Paclitaxel is present in a quantity by weight of at least 25 micrograms.

35. The stent according to claim 34, wherein Paclitaxel is present in a quantity by weight of at least 80 micrograms.

36. The stent according to claim 29, wherein FK506 is present in a quantity by weight of at least 60 micrograms.

37. The stent according to claim 36, wherein FK506 is present in a quantity by weight of at least 135 micrograms.
